(19) United States
(12) Patent Application Publication
(10) Pub. No.: US 2026/0266938 A1
(43) Pub. Date: Sep. 10, 2026

(54) SYSTEM AND METHOD FOR MAGNETIZATION PREPARED MAGNETIC RESONANCE IMAGING

(71) Applicant: Beth Israel Deaconess Medical Center, Inc., Boston, MA (US)

(72) Inventors: David ALSOP, Boston, MA (US); Manuel TASO, Boston, MA (US)

(21) Appl. No.: 19/163,662

(22) PCT Filed: Mar. 11, 2024
(86) PCT No.: PCT/US2024/019394

§ 371 Date: Sep. 9, 2025

Related U.S. Application Data
(60) Provisional application No. 63/489,359, filed on Mar. 9, 2023.
(60) Provisional application No. 63/510,239, filed on Jun. 26, 2023.

(51) Int. Cl. G01R 33/56; A61B 5/055; G01R 33/48; G01R 33/50; G01R 33/563

(52) U.S. Cl. CPC G01R 33/5602; G01R 33/4828; G01R 33/50; G01R 33/56366; A61B 5/055

(57) ABSTRACT

A method for magnetization prepared magnetic resonance imaging includes acquiring, using a magnetic resonance imaging (MRI) system, magnetic resonance (MR) data for at least two images of the region of interest of the subject. Each image of the at least two images is acquired using a different magnetization preparation or a different timing for magnetization preparation. The method further incudes generating the at least two images using the corresponding MR data and generating an image by comparing the at least two images. In some embodiments, the magnetization preparation is a T₂ preparation and the at least two images are used to generate an image with signal representing water exchange. In some embodiments, the magnetization preparation is a velocity selective preparation and the at least two images are used to generate an image with signal representing perfusion.

### CROSS-REFERENCE TO RELATED APPLICATIONS

This application is based on, claims priority to, and incorporates herein by reference in its entirety U.S. Ser. No. 63/489,359 filed Mar. 9, 2023, and entitled “System and Method for Imaging Tissue to Fluid Water Exchange by T2 Labeling,” and U.S. Ser. No. 63/510,239 filed Jun. 26, 2023, and entitled “System and Method for Imaging Tissue to Fluid Water Exchange by T2 Labeling.”

### STATEMENT REGARDING FEDERALLY SPONSORED RESEARCH

N/A

### FIELD

The present disclosure relates generally to magnetic resonance imaging and, more particularly, to systems and methods for magnetization prepared magnetic resonance imaging including, for example, for imaging tissue to fluid water exchange using T₂ labeling and for velocity selective arterial spin labeling imaging of tissue perfusion.

### BACKGROUND

The exchange of water between tissue spaces and surrounding fluids is a critical physiologic function. In the brain, water exchange at the choroid plexus and potentially other locations supports the generation and absorption of Cerebrospinal Fluid (CSF). The choroid plexus (CP) plays a key role in brain homeostasis and waste clearance as the main source of CSF production in the brain. The choroid plexus is a small structure sitting in the lateral ventricles and is not well characterized, including its dysfunction or impairment in several pathologies or just normal aging. Abnormal CSF production or absorption can lead to intercranial hypertension, hydrocephalus, and potentially dysfunction of the glymphatic drainage system of the brain that has been suggested as a potential cause of Alzheimer's Disease. In the kidneys, water exchange with the urine collecting system is critical to the maintenance of water balance and the concentration of urine. In both cases, the exchange of water is believed to be facilitated by special passive transporters in cell membranes called aquaporins. In the absence of aquaporins, water exchange through membranes is much slower such that tissue fluid boundaries with aquaporins tend to dominate exchange.

Currently, there are few methods available as standard of care for assessing water exchange. Radioactive water can be injected in animal studies but this is problematic for wide use. In the brain, phase contrast MRI can be used to measure the production of CSF, but the measurement is noisy and difficult because of the high pulsatility of CSF flow. Recently, researchers have demonstrated the ability to detect exchange of arterial blood labeled by arterial spin labeling into CSF spaces, for example, as discussed in Evans, P. et al. Non-Invasive MRI of Blood-Cerebrospinal Fluid Barrier Function. Nature Communications volume 11, Article number: 2081 (2020), Perera, C. et al. Pharmacological MRI with Simultaneous Measurement of Cerebral Perfusion and Blood-Cerebrospinal Fluid Barrier Function Using Interleaved Echo-Time Arterial Spin Labeling. Neuroimage. 2021 September; 238:118270, and Petitclerc, L. et al. Ultra-long-TE arterial spin labeling reveals rapid and brain-wide blood-to-CSF water transport in humans. Neuroimage. 2021 Dec. 15; 245:118755. A key insight was that the T₂ (transverse relaxation time) of fluid is very long, so images acquired with very long echo time (TE) contain fluid signals but the tissue signal decays away. The effect is very small in humans but suggested the possibility of imaging water exchange with noninvasive MRI methods. Another group suggested labeling of tissue water itself for imaging water exchange in animal models, instead of arterial labeling, by applying magnetization transfer (MT) pulses, for example as discussed in Li, A. M. et al. Age-dependent Cerebrospinal Flid-Tissue Water Exchange Detected by Magnetization Transfer Indirect Spin Labeling MRI. Magn Reson Med. 2022 May; 87 (5): 2287-2298. MT, however, requires high power and is less efficient at saturating tissue and especially blood than T₂ preparation. Water exchange is a contributor to the signal dynamics with bolus injection of MRI contrast but the effect is mixed with many others requiring complex and uncertain models. Other MRI techniques (e.g., arterial spin labeling (ASL)) that utilize contrast to image exchange or transport between tissues, between tissues and fluid, etc. can also face challenges.

### SUMMARY

In accordance with an embodiment, a method for magnetic resonance imaging of tissue to fluid water exchange in a region of interest in a subject includes acquiring, using a magnetic resonance imaging (MRI) system, magnetic resonance (MR) data for at least two images of the region of interest of the subject using a different T₂ preparation for each image, generating the at least two images using the corresponding MR data and generating an image with signal representing water exchange by comparing the at least two images.

In accordance with another embodiment, a method for magnetic resonance imaging of tissue to fluid water exchange in a region of interest in a subject includes acquiring, using a magnetic resonance imaging (MRI) system, magnetic resonance (MR) data for at least two images of the region of interest of the subject using a different timing for application of a T₂ preparation for each image, generating the at least two images using the corresponding MR data, and generating an image with signal representing water exchange by comparing the at least two images.

In accordance with another embodiment, a magnetic resonance imaging (MRI) system includes a magnet system configured to generate a polarizing magnetic field about at least a portion of a subject, a magnetic gradient system including a plurality of magnetic gradient coils configured to apply at least one magnetic gradient field to the polarizing magnetic field, a radio frequency (RF) system configured to apply an RF field to the subject and to receive magnetic resonance signals from the subject using a coil array, and a computer system. The computer system is programmed to acquire magnetic resonance (MR) data for at least two images of the region of interest of the subject using a different T₂ preparation for each image, generate the at least two images using the corresponding MR data, and generate an image with signal representing water exchange by comparing the at least two images.

In accordance with another embodiment, a magnetic resonance imaging (MRI) system includes a magnet system configured to generate a polarizing magnetic field about at least a portion of a subject, a magnetic gradient system including a plurality of magnetic gradient coils configured to apply at least one magnetic gradient field to the polarizing magnetic field, a radio frequency (RF) system configured to apply an RF field to the subject and to receive magnetic resonance signals from the subject using a coil array, and a computer system. The computer system is programmed to acquire magnetic resonance (MR) date for at least two images of the region of interest of the subject using a different timing for application of a T₂ preparation for each image, generate the at least two images using the corresponding MR data, generate an image with signal representing water exchange by comparing the at least two images.

In accordance with another embodiment, a method for velocity selective arterial spin labeling magnetic resonance imaging of tissue perfusion in a region of interest in a subject includes acquiring, using a magnetic resonance imaging (MRI) system, magnetic resonance (MR) data for at least two images of the region of interest of the subject using a different timing for application of a velocity selective preparation for each image, generating the at least two images using the corresponding MR data, and generating an image with signal representing perfusion by comparing the at least two images.

### BRIEF DESCRIPTION OF THE DRAWINGS

The present disclosure will hereafter be described with reference to the accompanying drawings, wherein like reference numerals denote like elements.

FIG. 1 is a block diagram of an example magnetic resonance imaging (MRI) system in accordance with an embodiment;

FIG. 2 illustrates a method for magnetic resonance imaging of tissue to fluid water exchange in a region of interest in a subject in accordance with an embodiment;

FIG. 3 illustrates an example T₂-prepared inversion recovery pulse sequence in accordance with an embodiment;

FIG. 4 illustrates a method for magnetic resonance imaging of tissue to fluid water exchange in a region of interest in a subject in accordance with an embodiment;

FIG. 5 illustrates an example T₂-prepared, long echo time inversion recovery pulse sequence with a T₂-preparation module applied before an inversion recovery pulse in accordance with an embodiment;

FIG. 6 illustrates an example T₂-prepared, long echo time inversion recovery pulse sequence with a T₂-preparation module applied after an inversion recovery pulse in accordance with an embodiment;

FIG. 7 shows an example graph illustrating a derivative with respect to 1/T₁ versus a time of the end of T₂-preparation in accordance with an embodiment;

FIG. 8 shows an example graph illustrating a derivative with respect to 1/T₁ versus a time of the end of T₂-preparation in accordance with an embodiment;

FIG. 9 shows an example graph illustrating sensitivity to water exchange as a function of a time of the end of T₂-preparation in accordance with an embodiment;

FIG. 10 illustrates an example magnetization preparation sequence in accordance with an embodiment;

FIG. 11 illustrates an example magnetization prepared inversion recovery pulse sequence configured to include a plurality of inversion recovery (IR) pulses in accordance with an embodiment; and

FIG. 12 illustrates an example magnetization prepared inversion recovery pulse sequence configured to include a plurality of inversion recovery (IR) pulses in accordance with an embodiment;

FIG. 13 illustrates a method for velocity selective arterial spin labeling magnetic resonance imaging of tissue perfusion in a region of interest in a subject in accordance with an embodiment;

FIG. 14A shows an example graph illustrating magnetization as a function of time before readout for acquisitions with and without T₂ preparation in accordance with an embodiment;

FIG. 14B shows an example graph illustrating magnetization difference between a tissue and a fluid with and without T₂ preparation in accordance with an embodiment;

FIG. 15 is a block diagram of an example computer system in accordance with an embodiment.

### DETAILED DESCRIPTION

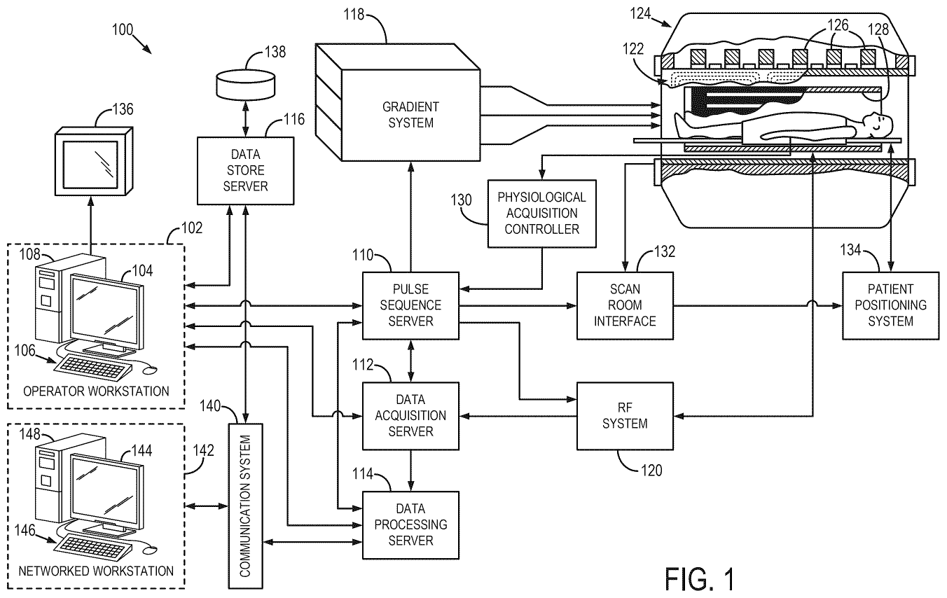

Referring now to FIG. 1, the disclosed systems and methods may be implemented using or designed to accompany a magnetic resonance imaging (“MRI”) system 100, such as is illustrated in FIG. 1. The MRI system 100 includes an operator workstation 102, which will typically include a display 104, one or more input devices 106 (such as a keyboard and mouse or the like), and a processor 108. The processor 108 may include a commercially available programmable machine running a commercially available operating system. The operator workstation 102 provides the operator interface that enables scan prescriptions to be entered into the MRI system 100. In general, the operator workstation 102 may be coupled to multiple servers, including a pulse sequence server 110; a data acquisition server 112; a data processing server 114; and a data store server 116. The operator workstation 102 and each server 110, 112, 114, and 116 are connected to communicate with each other. For example, the servers 110, 112, 114, and 116 may be connected via a communication system 140, which may include any suitable network connection, whether wired, wireless, or a combination of both. As an example, the communication system 140 may include proprietary networks, dedicated networks, as well as open networks, such as the internet.

The pulse sequence server 110 functions in response to instructions downloaded from the operator workstation 102 to operate a gradient system 118 and a radiofrequency (“RF”) system 120. Gradient waveforms to perform the prescribed scan are produced and applied to the gradient system 118, which excites gradient coils in an assembly 122 to produce the magnetic field gradients Gₓ, G_{y}, G_{z} used for position encoding magnetic resonance signals. The gradient coil assembly 122 forms part of a magnet assembly 124 that includes a polarizing magnet 126 and a whole-body RF coil 128.

RF waveforms are applied by the RF system 120 to the RF coil 128, or a separate local coil (not shown in FIG. 1), in order to perform the prescribed magnetic resonance pulse sequence. Responsive magnetic resonance signals detected by the RF coil 128, or a separate local coil, are received by the RF system 120, where they are amplified, demodulated, filtered, and digitized under direction of commands produced by the pulse sequence server 110. The RF system 120 includes an RF transmitter for producing a wide variety of RF pulses used in MRI pulse sequences. The RF transmitter is responsive to the scan prescription and direction from the pulse sequence server 110 to produce RF pulses of the desired frequency, phase, and pulse amplitude waveform. The generated RF pulses may be applied to the whole-body RF coil 128 or to one or more local coils or coil arrays.

The RF system 120 also includes one or more RF receiver channels. Each RF receiver channel includes an RF preamplifier that amplifies the magnetic resonance signal received by the coil 128 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

$$M = \sqrt{{I}^{2} + {Q}^{2}} \qquad ( 1 )$$

and the phase of the received magnetic resonance signal may also be determined according to the following relationship:

$$φ = {tan}^{- 1} ( \frac{Q}{I} ) \qquad ( 2 )$$

The pulse sequence server 110 also optionally receives patient data from a physiological acquisition controller 130. By way of example, the physiological acquisition controller 130 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized magnetic resonance signal samples produced by the RF system 120 are received by the data acquisition server 112. The data acquisition server 112 operates in response to instructions downloaded from the operator workstation 102 to receive the real-time magnetic resonance data and provide buffer storage, such that no data is lost by data overrun. In some scans, the data acquisition server 112 does little more than pass the acquired magnetic resonance data to the data processor server 114. However, in scans that require information derived from acquired magnetic resonance data to control the further performance of the scan, the data acquisition server 112 is programmed to produce such information and convey it to the pulse sequence server 110. For example, during prescans, magnetic resonance data is acquired and used to calibrate the pulse sequence performed by the pulse sequence server 110. As another example, navigator signals may be acquired and used to adjust the operating parameters of the RF system 120 or the gradient system 118, or to control the view order in which k-space is sampled. In still another example, the data acquisition server 112 may also be employed to process magnetic resonance signals used to detect the arrival of a contrast agent in a magnetic resonance angiography (“MRA”) scan. By way of example, the data acquisition server 112 acquires magnetic resonance data and processes it in real-time to produce information that is used to control the scan.

The data processing server 114 receives magnetic resonance data from the data acquisition server 112 and processes it in accordance with instructions downloaded from the operator workstation 102. Such processing may, for example, include one or more of the following: reconstructing two-dimensional or three-dimensional images by performing a Fourier transformation of raw k-space data; performing other image reconstruction techniques, such as iterative or back-projection reconstruction techniques; applying filters to raw k-space data or to reconstructed images; generating functional magnetic resonance images; calculating motion or flow images; and so on.

Images reconstructed by the data processing server 114 are conveyed back to the operator workstation 102. Images may be output to operator display 112 or a display 136 that is located near the magnet assembly 124 for use by attending clinician. Batch mode images or selected real time images are stored in a host database on disc storage 138. When such images have been reconstructed and transferred to storage, the data processing server 114 notifies the data store server 116 on the operator workstation 102. The operator workstation 102 may be used by an operator to archive the images, produce films, or send the images via a network to other facilities.

The MRI system 100 may also include one or more networked workstations 142. By way of example, a networked workstation 142 may include a display 144, one or more input devices 146 (such as a keyboard and mouse or the like), and a processor 148. The networked workstation 142 may be located within the same facility as the operator workstation 102, or in a different facility, such as a different healthcare institution or clinic. The networked workstation 142 may include a mobile device, including phones or tablets.

The networked workstation 142, whether within the same facility or in a different facility as the operator workstation 102, may gain remote access to the data processing server 114 or data store server 116 via the communication system 140. Accordingly, multiple networked workstations 142 may have access to the data processing server 114 and the data store server 116. In this manner, magnetic resonance data, reconstructed images, or other data may exchange between the data processing server 114 or the data store server 116 and the networked workstations 142, such that the data or images may be remotely processed by a networked workstation 142. This data may be exchanged in any suitable format, such as in accordance with the transmission control protocol (“TCP”), the internet protocol (“IP”), or other known or suitable protocols.

The present disclosure describes systems and methods for magnetization prepared magnetic resonance imaging (MRI). In some embodiments, the disclosed techniques for magnetization prepared MRI can be used for systems and methods for velocity selective arterial spin labeling magnetic resonance imaging of tissue perfusion. For example, two or more images of a region of interest in a subject can be acquired using a different timing for application of a velocity selective preparation for each image. An image with signal representing perfusion can be generated by comparing the at least two images.

In some embodiments, the disclosed techniques for magnetization prepared MRI can be used for systems and methods for magnetic resonance imaging of tissue to fluid water exchange in a region of interest in a subject where the imaging of the water exchange is based on the difference in transverse relaxation (T₂) between fluid and tissue/blood components. Advantageously, a T₂ preparation may be used to impart sensitivity to tissue to fluid water exchange. In some embodiments, at least two images of the region of interest of the subject may be acquired using an MRI system. Each of the at least two images can be acquired using a different T₂ preparation (e.g., with or without a T₂ preparation, different duration T₂ preparation) or a different timing for the T₂ preparation. An image with signal representing water exchange can be generated by comparing the at least two images (e.g., by subtraction, fitting to a model, or other comparison algorithm). In some embodiments, the disclosed systems and methods can provide an approach for sensitive measurement of water exchange from, for example, short to long echo time (TE) compartments to assess water exchange within a region of interest.

In some embodiments, a T₂ preparation may be applied at a predetermined time before the acquisition (i.e., readout) of a first image (e.g., a heavily weighted T₂ image). As used herein, the first image acquired using a T₂ preparation may also be referred to as a label image. A second image may be acquired and compared to the first image. As used herein, the second image may also be referred to as a control image. In some embodiments, the second image may be acquired with a different T₂ preparation (e.g., without a T₂ preparation (the T₂ preparation is turned off), different duration T₂ preparation). In some embodiments, the second image may be acquired using a T₂ preparation that is applied at a different time than the T₂ preparation for the first image is applied. The comparison of the first image and the second image can be used to infer the water exchange that has occurred between the tissue and fluid. In some embodiments, the comparison may be used to generate an image with signal representing water exchange.

In some embodiments, the acquisition of the first and second images also includes application (or playing out) of one or more inversion recovery (IR) pulses. Inversion recovery can approximately null the fluid signal, for example, as employed in a Fluid Attenuated Inversion Recovery (FLAIR) pulse sequence. With the fluid signal greatly attenuated by inversion recovery, motion related and other artifacts of the otherwise large fluid signal can be greatly reduced. In some embodiments, where the first and second images are both acquired with a T₂ preparation, the T₂ preparation for the first image can be applied at an optimal time before the IR pulse(s) and the T₂ preparation for the second image can be applied at an optimal time after the IR pulse(s). Acquiring two images with the T₂ preparation applied before (in the first image) the IR pulse(s) and after (in the second image) the IR pulse(s) can, for example, give equal sensitivity to systematic effects of imperfect T₂ preparation and variable T₂ in fluid and can allow for control of systematic errors in the T₂ preparation. In some embodiments, the timing of the inversion pulses can be selected (e.g., optimized) to reduce systematic errors over a range of T₁'s and T₂'s. In some embodiments, comparison of the two images can be performed by subtracting the two images which can give a signal reflective of water exchange. In some embodiments, dividing the subtraction of the two images by a reference image without inversion recovery or T₂ preparation can provide a semiquantitative ratio image. In some embodiments, the method can be configured to reduce the sensitivity to variable longitudinal magnetizations (T₁'s) of the fluid. In some embodiments, changing the repetition time (TR) of one of the two images versus the other can reduce the T₁ sensitivity of the measurement and having a different TR for the second (or control) image can reduce potential errors from differences in fluid T₁. For example, the TR of the second (or control) image can be different (e.g., longer) than the TR of the first (or label) image.

In some embodiments, quantification of water exchange may also be performed using the image with signal representing water exchange generated by the comparison of the two images. A model for contribution of tissue-fluid water exchange to the MRI signal may be used for quantification. In some embodiments, the semiquantitative ratio can be related to the exchange rate.

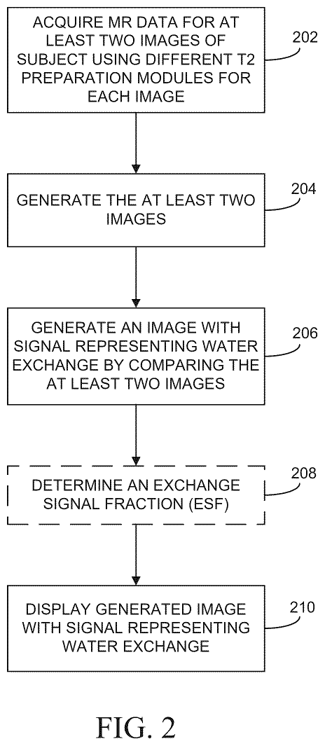

In some embodiments, the region of interest in which the tissue to fluid water exchange is imaged can be, for example, the brain or the kidneys. In one example, the disclosed systems and methods may be used for quantitative assessment of intrarenal fluid exchange which may serve as a biomarker for diagnosis and prognosis of renal diseases. In addition, the disclosure systems and methods can provide for a non-invasive assessment of renal filtration and water exchange that may complement other measures of renal function. In another example, the disclosed systems and methods can be used for in vivo studies of CSF exchange that may reflect the changes in glymphatic clearance or CSF production with aging, Alzheimer's disease, intracranial hypertension and other disorders. While the following description of FIGS. 2-10 may be discussed in terms of an example application of imaging water exchange between tissue and fluid in the brain (e.g., water exchange between the choroid plexus (CP) and cerebral spinal fluid (CSF)), it should be understood that the disclosed systems and methods described herein may be used for imaging water exchange for any region of the anatomy of a subject.

FIG. 2 illustrates a method for magnetic resonance imaging of tissue to fluid water exchange in a region of interest in a subject in accordance with an embodiment. Although the blocks of the process in FIG. 2 are illustrated in a particular order, in some embodiments, one or more blocks may be executed in a different order than illustrated in FIG. 2 or may be bypassed.

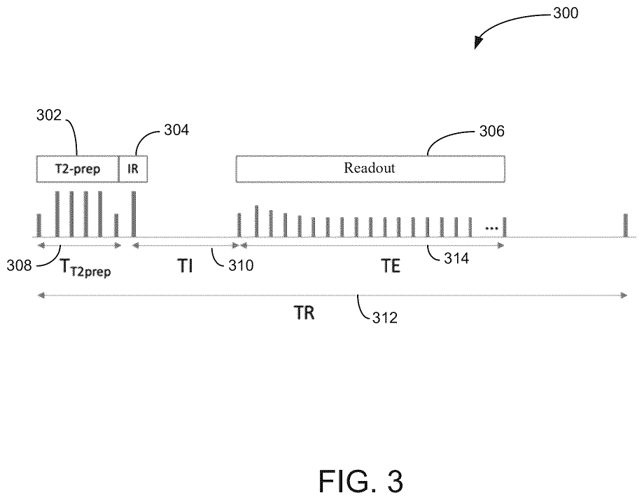

At block 202, MR data for at least two images may be acquired from a region of interest of a subject using, for example, an MRI system (e.g., MRI system 100 shown in FIG. 1). The MR data for each image may be acquired using a pulse sequence performed on the MRI system and the MR data for each image may be acquired with a different T₂ preparation. For example, in some embodiments, MR data for a first image (or label image) may be acquired using a T₂ preparation applied at a predetermined time before the acquisition (i.e., readout) and MR data for a second image (or control image) may be acquired without T₂ preparation (e.g., the T₂ preparation is turned off). In some embodiments, the MR data for each image may be acquired with a T₂ preparation with a different duration. While the following discussion of FIGS. 2-4 will refer to the acquisition and reconstruction of two images, it should be understood that in some embodiments, more than two images may be acquired, reconstructed, and then compared to generate an image with signal representing water exchange. As mentioned, in some embodiments, MR data for the first image may be acquired from a subject using a pulse sequence that includes a T₂ preparation module applied at a predetermined time before a readout. In some embodiments, the T₂ preparation module may be implemented using known pulses and techniques for T₂ preparation. In one example, the T₂ preparation module (e.g., T₂ preparation module 302 shown in FIG. 3) can include a 90 degree hard pulse followed by four adiabatic hyperbolic secant refocusing pulses and one final 90 degree hard pulse. The readout of the pulse sequence may be, for example, a three-dimensional fast spin echo readout. In some embodiments, the pulse sequence may also include one or more inversion recovery pulses applied a predetermined time after the T₂ preparation. FIG. 3 illustrates an example T₂-prepared inversion recovery pulse sequence in accordance with an embodiment. The pulse sequence 300 shown in FIG. 3, includes a T₂ preparation module 302 with a duration T_{T2prep} 308, an IR pulse 304, a readout 306 with an echo time TE 314, an inversion time 310 and a repetition time 312. In some embodiments, the echo time TE 314 may be a long echo time. While FIG. 3 shows one IR pulse 304, it should be understood that in some embodiments the pulse sequence 300 can include a plurality of IR pulses 304.

Returning to FIG. 2, at block 202, MR data for a second image (or control image) may be acquired from a subject using a pulse sequence that includes a different T₂ preparation, for example, MR data for the second image can be acquired with a pulse sequence that does not include a T₂ preparation module (e.g., pulse sequence 300 shown in FIG. 3 without the T₂ preparation module 302) or MR data for the second image may be acquired with a pulse sequence using a T₂ preparation with a different duration (e.g., pulse sequence 300 shown in FIG. 3 with a different duration 308 for the T₂ preparation module 302) than the pulse sequence for the first image. The readout may be, for example, a three-dimensional fast spin echo readout. In some embodiments, the pulse sequence may also include one or more inversion recovery pulses applied a predetermined time before the readout. The MR data acquired for the at least two images, e.g., a first image and a second image, can be stored in, for example, data storage of an MR system (e.g., MRI system 100 shown in FIG. 1) or data storage of other computer systems.

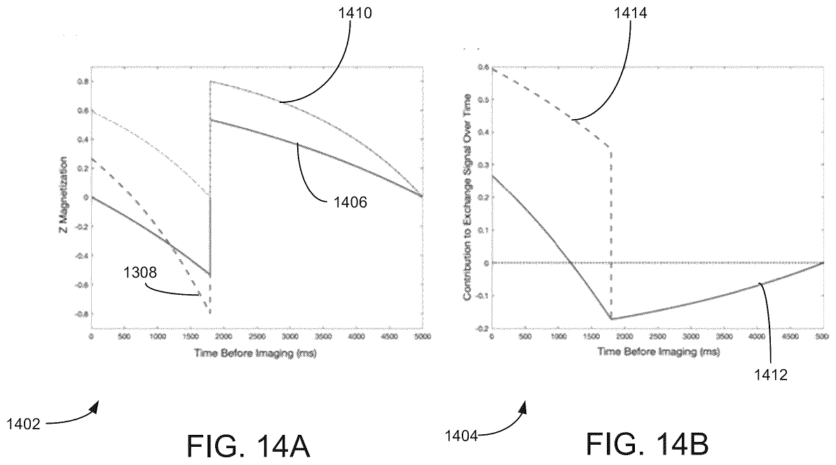

At block 204, the at least two images, e.g., a first image and a second image, may be generated (or reconstructed) based on the corresponding MR data for each image acquired at block 202 using known reconstruction methods. In some embodiments, the first image and the second image may be reconstructed using, for example, a data processing server 114 of an MRI system 100. In some embodiments, the first image and second image can be reconstructed using a computer system (e.g. computer system 1400 shown in FIG. 14) configured to access or receive the MR data acquired by the MR system. The generated first image and second image may be stored in, for example, data storage of an MR system (e.g., MRI system 100 shown in FIG. 1) or data storage of other computer systems.

At block 206, an image with signal representing water exchange may be generated by comparing the at least two images acquired at block 202 and generated at block 204, for example, comparing a first image and a second image. In some embodiments, the comparison may be implemented by subtracting the second image from the first image. In some embodiments, the comparison may be implemented by fitting the first and second images to a model or by using other comparison algorithms. The image with signal representing water exchange generated at block 206 may be stored in, for example, data storage of an MR system (e.g., MRI system 100 shown in FIG. 1) or data storage of other computer systems. At block 208, the image with signal representing water exchange may be used to determine an exchange signal fraction. At block 210, the generated image with signal representing water exchange may be displayed on a display, for example, a display of an MRI system (e.g., displays 104, 136 and/or 144 of MRI system 100 shown in FIG. 1) or a display of other computer systems.

The movement of water from one compartment to another is potentially of great interest for understanding physiology and pathophysiology, testing drug treatments, and potentially the diagnosis of individual patients. Water exchange can also be a key function for systems requiring the transfer of water for their function. In the choroid plexus, and potentially other boundaries of the central nervous system, exchange of water with the CSF can be key for the production of CSF. This production can be of importance for maintaining CSF pressure and potentially for the clearance of larger molecules from the brain through a process termed glymphatics. In the kidney, water exchange can be integral to the control of water balance through exchange with the collecting system that leads to the production of urine.

As discussed above, MRI can be made sensitive to water exchange if it is possible to selectively attenuate, or label, one compartment and then selectively image the other compartment. Because there is typically a substantial offset to the measurement, the labeled image can be compared with a second image that does not label the other compartment. Simply not labeling for the control image (as discussed above with respect to FIG. 2), however, can be insufficient because the labeling is not perfectly selective and instead more weakly attenuates the imaged compartment.

As mentioned above, in some embodiments rather than an image without T₂ preparation or a different T₂ preparation duration, at least two images may be acquired with T₂ preparations of different timing to vary the sensitivity to water exchange. In these embodiments, each acquired image can use the same T₂ labeling strategy but applied at different times. Since the water exchange effect can build up over time, the difference between the images will still be sensitive to water exchange. However, equal saturation of the images pool at different times can lead to different effects due to the relaxation and recovery times of MRI. To overcome this limitation, in some embodiments, the label and control preparations can be applied on opposite sides of an inversion recovery pulse. In some embodiments, with suitable optimization of timing, this strategy can control for a reasonable range of saturation and relaxation parameters.

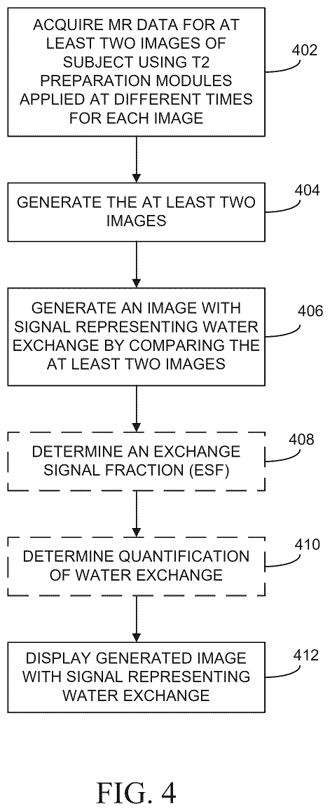

FIG. 4 illustrates a method for magnetic resonance imaging of tissue to fluid water exchange in a region of interest in a subject in accordance with an embodiment. Although the blocks of the process in FIG. 4 are illustrated in a particular order, in some embodiments, one or more blocks may be executed in a different order than illustrated in FIG. 4 or may be bypassed.

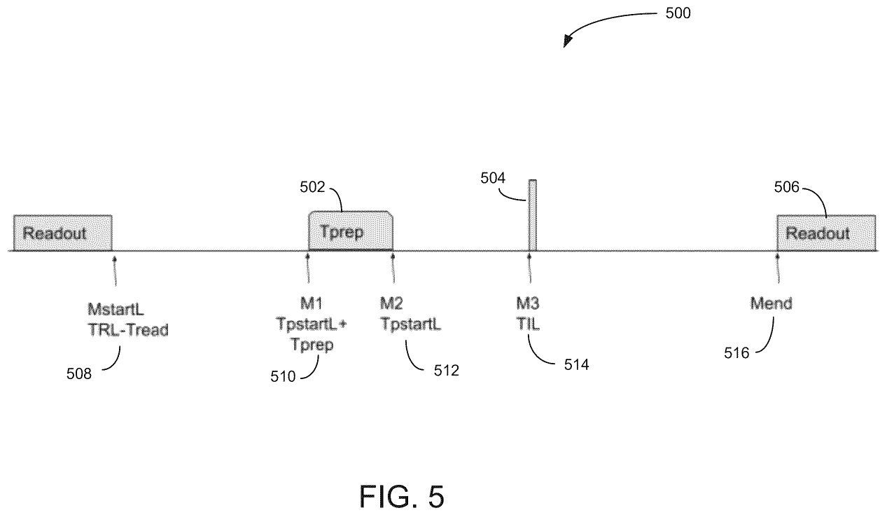

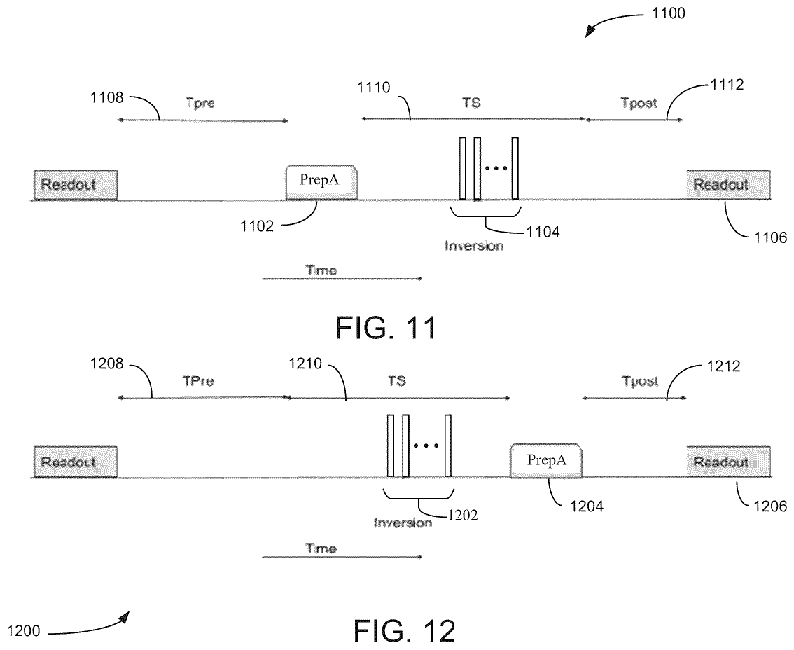

At block 402, MR data for at least two images may be acquired from a region of interest of a subject using, for example, an MRI system (e.g., MRI system 100 shown in FIG. 1). The MR data for each image may be acquired using a pulse sequence performed on the MRI system and the MR data for each image may be acquired with a different timing for the application of a T₂ preparation. While the following discussion of FIGS. 4-9 will refer to the acquisition and reconstruction of two images (e.g., a label image and a control image), it should be understood that in some embodiments, more than two images may be acquired, reconstructed, and then compared to generate an image with signal representing water exchange. In some embodiments, for example, MR data for a first (or label) image may be acquired from a subject using a pulse sequence that includes a T₂ preparation module applied at a first predetermined time before a readout. In some embodiments, the first pulse sequence can include at least one inversion recovery pulse and the T₂ preparation can be applied at a first time before the IR pulse(s). In some embodiments, the T₂ preparation module may be implemented using known pulses and techniques for T₂ preparation. In some embodiments, the preparation module can be any pulse that imparts a multiplicative change on longitudinal magnetization. The readout may be, for example, a three-dimensional fast spin echo readout. FIG. 5 illustrates an example T₂-prepared, long echo time inversion recovery pulse sequence with a T₂-preparation module applied before an inversion recovery pulse in accordance with an embodiment. The pulse sequence 500 shown in FIG. 5, includes a T₂ preparation module 502, an IR pulse 504, and a readout 506. As used herein with regard to FIGS. 5-9, the suffix “L” is used to refer to the label image acquisition and the suffix “C” is used to refer to the control image acquisition (described further below) for all parameters. In FIG. 5, increasing time moves from left to right. The sequence 500 includes various magnetization and timing including, for example, a start time for the acquisition TR_{L}−T_{read} (508), a start time for the T₂ preparation, T_{pstartL}+Tₚᵣₑₚ (510), an end time for the T₂ preparation, T_{pstartL} (512), an inversion recovery time TI_{L} (514), and an end of magnetization, M_{end} (516). The labeling T₂ preparation 502 can be applied for a time period Tₚᵣₑₚ. In some embodiments, the preparation module 502 can be any pulse that imparts a multiplicative change on longitudinal magnetization. In some embodiments the preparation module 502 can be configured to convert the magnetization. While only one IR pulse 504 is shown in FIG. 5, it should be understood that in some embodiments more than one IR pulse 504 may be included after the T₂ preparation module 502 and before the readout 506, as discussed further below with respect to FIG. 11.

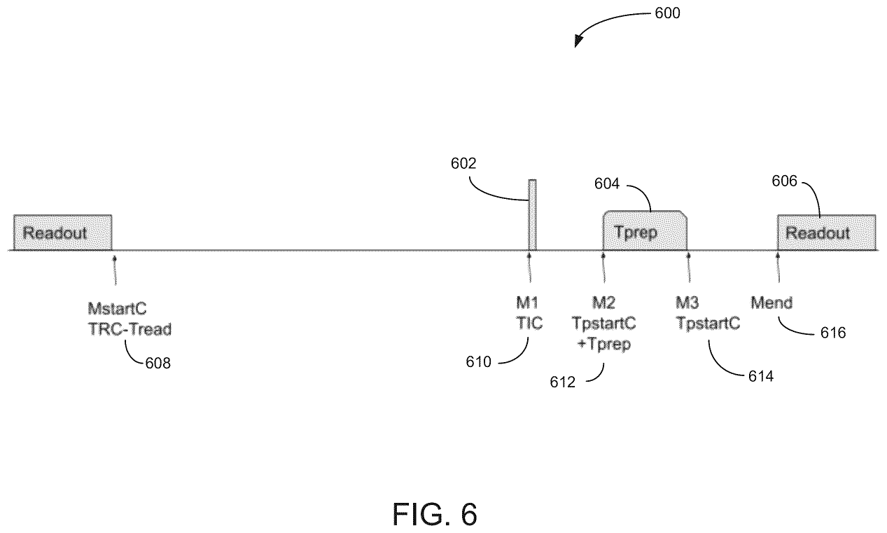

Returning to FIG. 4, at block 402, MR data for a second image (or control image) may be acquired from a region of interest of a subject using a pulse sequence that includes a T₂ preparation module applied at a second predetermined time before a readout where the second time is different than the first time. In some embodiments, the second pulse sequence can include at least one inversion recovery pulse and the T₂ preparation can be applied at a second time after the IR pulse(s) and before the readout. In some embodiments, the T₂ preparation module may be implemented using known pulses and techniques for T₂ preparation. In some embodiments, the preparation module can be any pulse that imparts a multiplicative change on longitudinal magnetization. The readout may be, for example, a three-dimensional fast spin echo readout. FIG. 6 illustrates an example T₂-prepared, long echo time inversion recovery pulse sequence with a T₂-preparation module applied after an inversion recovery pulse in accordance with an embodiment. In FIG. 6, increasing time moves from left to right. The pulse sequence 600 shown in FIG. 6, includes. An IR pulse 602, a T₂ preparation module 604, and a readout 606. The sequence 600 also includes various magnetization and timing including, for example, a start time for the acquisition TR_{C}−T_{read} (608), a start time for the T₂ preparation, T_{pstartC}+Tₚᵣₑₚ (612), an inversion recovery time TI_{C} (610), an end time for the T₂ preparation, T_{pstartC} (614), and an end of magnetization, M_{end} (616). While only one IR pulse 602 is shown in FIG. 6, it should be understood that in some embodiments more than one IR pulse 602 may be included before the T₂ preparation module 604, as discussed further below with respect to FIG. 12.

Referring to both FIGS. 5 and 6, while M_{end} (516, 616) should be equal for both image acquisitions, in principle, TR and Mₛₜₐᵣₜ can be different for the label and the control images. T_{read} can likely be equal because using identical imaging sequences will match image parameters across spatial encoding. In some embodiments, there can be advantages to making Mₛₜₐᵣₜ effectively zero by applying a nonselective saturation to both compartments since the saturation effect of the imaging is typically imperfect and sensitive to parameters such as the RF field amplitude. There can also be advantages to keeping M_{end} small, since it can typically be difficult to measure a small signal in the presence of a larger background.

In some embodiments, the acquisition of the label and control images at block 402 can assume selective imaging of the target compartments (e.g., a tissue such as CP and a fluid such as CSF). For CSF imaging by T₂ selectivity, this can be efficiently achieved by very long TE imaging, as with, for example, a multiple spin echo sequence.

In some embodiments, for the acquisition of the first (or label) image at block 402, it can be assumed that there is no z recovery, only multiplicative attenuation or potentially even inversion during preparation. In some embodiments, this effect can be described by the factor αₚᵣₑₚ with value between −1 to 1 which is discussed further below. As used here, T_{pstartL} can be defined as the time after the end of the preparation for the acquisition of the first or label image.

In some embodiments, the inversion time (TI_{L} (514), shown in FIG. 5) for the acquisition of the first or label image can be determined by following the magnetization evolution according to the Bloch equations:

$${M}_{1} = 1 - ( 1 - {M}_{startL} ) {e}^{- \frac{( TRL - {T}_{r e a d} - {τ}_{p s t a r t L} - {T}_{prep} )}{{T}_{1}}} \qquad ( 3 )$$

$${M}_{2} = [ 1 - ( 1 - {M}_{staartL} ) {e}^{- \frac{( TRL - {T}_{r e a d} - {T}_{p s t a r t L} - {T}_{prep} )}{{T}_{1}}} ] {α}_{p r e p} \qquad ( 4 )$$

$${M}_{3} = 1 - {e}^{\frac{( {T}_{pstartL} - TIL}{{T}_{1}}} + [ 1 - ( 1 - {M}_{startL} ) {e}^{- \frac{( TRL - Tread - {T}_{pstartL} - {T}_{prep} )}{{T}_{1}}} ] {α}_{prep} {e}^{- \frac{( {T}_{pstartL - TIL} )}{{T}_{1}}} \qquad ( 5 )$$

$${M}_{e n d} = [ 1 - 2 {e}^{- \frac{TIL}{{T}_{1}}} + {e}^{- \frac{{T}_{p startL}}{{T}_{1}}} ( 1 - {α}_{prep} ) + [ ( 1 - {M}_{startL} ) {e}^{- \frac{( TRL - {T}_{r e a d} - {T}_{prep} )}{{T}_{1}}} {α}_{prep} ] ] \qquad ( 6 )$$

If a particular M_{end} is targeted, this equation can be inverted to solve for TI_{L} (514):

$$TIL = - T 1 ln [ \frac{1}{2} [ 1 - {M}_{e n d} + {e}^{- \frac{{T}_{p s t a a r t L}}{{T}_{1}}} ( 1 - {α}_{prep} ) + [ ( 1 - {M}_{startL} ) {e}^{- \frac{( TRL - {T}_{r e a d} - {T}_{prep} )}{{T}_{1}}} {α}_{prep} ] ] ] \qquad ( 7 )$$

In some embodiments, it is likely that αₚᵣₑₚ and T1 are not known with high accuracy because the relaxation times can vary across regions, imperfection in RF fields may alter preparation efficiency, etc. It may be desirable to reduce, minimize or otherwise optimize sensitivity to variations in these parameters. To first order, these can be determined by their first derivatives, which can be calculated as follows.

$$\frac{{dM}_{endL}}{d {α}_{prep}} = ( 1 - {M}_{startL} ) {e}^{- \frac{( TRL - {T}_{read} - {T}_{prep} )}{{T}_{1}}} - {e}^{- \frac{{T}_{pstartL}}{{T}_{1}}} \qquad ( 8 )$$

$$\frac{{dM}_{endL}}{d ( 1 / {T}_{1} )} = - [ - 2 TIL {e}^{- \frac{TIL}{{T}_{1}}} + {T}_{pstartL} {e}^{- \frac{{T}_{pstartL}}{{T}_{1}}} ( 1 - {α}_{prep} ) + ( TRL - {T}_{read} - {T}_{prep} ) [ ( 1 - {M}_{startL} ) {e}^{- \frac{( TRL - {T}_{read} - {T}_{prep} )}{{T}_{1}}} {α}_{prep} ] ] \qquad ( 9 )$$

In some embodiments, for the acquisition of the second (or control) image at block 402 it can be assumed that there is no z recovery, only T₂ decay during preparation. In some embodiments, T_{pstartC} can be defined as the time of the end of the T₂ prep for the acquisition of the second or control image. This way, T_{pstartC}=0 can correspond to T₂ prep applied just before imaging of the control image.

Following the evolution of magnetization according to the Bloch equations:

$${M}_{1} = 1 - ( 1 - {M}_{startC} ) {e}^{- \frac{( TRC - {T}_{read} - TIC )}{{T}_{1}}} \qquad ( 10 )$$

$${M}_{2} = 1 - {e}^{- \frac{( TIC - {T}_{pstartC} - {T}_{prep} )}{{T}_{1}}} - [ 1 - ( 1 - {M}_{startC} ) {e}^{- \frac{( TRC - {T}_{read} - TIC )}{{T}_{1}}} ] {e}^{- \frac{( TIC - {T}_{pstartC} - {T}_{prep} )}{{T}_{1}}} \qquad ( 11 )$$

$${M}_{3} = [ 1 - 2 {e}^{- \frac{( TIC - {T}_{pst} - {T}_{prep} )}{{T}_{1}}} + [ ( 1 - {M}_{startC} ) {e}^{- \frac{TRC - {T}_{read} - TI}{{T}_{1}}} ] {e}^{- \frac{( TIC - {T}_{pst} - {T}_{prep} )}{{T}_{1}}} ] {e}^{- \frac{{T}_{prep}}{{T}_{2}}} \qquad ( 12 )$$

$${M}_{endC} = 1 - {e}^{- \frac{{T}_{pstartC}}{{T}_{1}}} ( 1 - {α}_{prep} ) - [ 2 {e}^{- \frac{( TIC - {T}_{prep} )}{{T}_{1}}} - ( 1 - {M}_{startC} ) {e}^{- \frac{TRC - {T}_{read} - {T}_{prep}}{{T}_{1}}} ] {α}_{prep} \qquad ( 13 )$$

$$TIC = - T 1 ln [ \frac{1}{2} [ ( 1 - {M}_{end} ) {e}^{- \frac{{T}_{prep}}{{T}_{1}}} {α}_{prep} - {e}^{- \frac{{T}_{pstartC} + {T}_{prep}}{{T}_{1}}} ( {α}_{prep} = 1 ) + [ ( 1 - {M}_{startC} ) {e}^{- \frac{TRC - {T}_{read}}{{T}_{1}}} ] ] ] \qquad ( 14 )$$

$$\frac{{dM}_{endC}}{d {α}_{prep}} = {e}^{- \frac{pstartC}{{T}_{1}}} - [ 2 {e}^{- \frac{( TIC - {T}_{prep} )}{{T}_{1}}} - ( 1 - {M}_{startC} ) {e}^{- \frac{TRC - {T}_{read} - {T}_{prep}}{{T}_{1}}} ] \qquad ( 15 )$$

$$\frac{{dM}_{endC}}{d ( 1 / {T}_{1} )} = {T}_{pstartC} {e}^{- \frac{{T}_{pstartC}}{{T}_{1}}} ( 1 - {α}_{prep} ) + [ 2 ( TIC - {T}_{prep} ) {e}^{- \frac{( TIC - {T}_{prep} )}{{T}_{1}}} - ( TRC - {T}_{read} - {T}_{prep} ) ( 1 - {M}_{startC} ) ] \qquad ( 16 )$$

In some embodiments, it may be desirable to match the acquisition (i.e., sequence 500) for the label image to the acquisition (i.e. sequence 600) of the control image for sensitivity to αₚᵣₑₚ. In some embodiments, to match the two acquisitions, M_{end} may first be required to be matched between the two sequences (500, 600). These equations do not consider the exchange effect so any difference would be a systematic error. Next, the derivative with respect to αₚᵣₑₚ can be matched.

For the label image acquisition:

$$\frac{{dM}_{end}}{d {α}_{prep}} = ( 1 - {M}_{startL} ) {e}^{- \frac{( TRL - {T}_{read} - {T}_{prep} )}{{T}_{1}}} {e}^{- \frac{{T}_{pstartC}}{{T}_{1}}} \qquad ( 17 )$$

For the control image acquisition:

$$\frac{{dM}_{endC}}{d {α}_{prep}} = {e}^{- \frac{{T}_{pstartC}}{{T}_{1}}} - [ 2 {e}^{- \frac{( TIC - {T}_{prep} )}{{T}_{1}}} - ( 1 - {M}_{staartC} ) {e}^{- \frac{TRC - {T}_{read} - {T}_{prep}}{{T}_{1}}} ] \qquad ( 18 )$$

In some embodiments, this can be simplified by inserting from the equation for M_{end}

$${M}_{endC} = 1 - {e}^{- \frac{{T}_{pstartC}}{{T}_{1}}} ( 1 - {α}_{prep} ) - [ 2 {e}^{- \frac{( TIC - {T}_{prep} )}{{T}_{1}}} - ( 1 - {M}_{startC} ) {e}^{- \frac{TRC - {T}_{read} - {T}_{prep}}{{T}_{1}}} ] {α}_{prep} \qquad ( 19 )$$

$$Or [ {M}_{endC} + {e}^{- \frac{{T}_{pstartC}}{{T}_{1}}} - 1 ] \frac{1}{{α}_{prep}} = {e}^{- \frac{{T}_{pstartC}}{{T}_{1}}} - [ 2 {e}^{- \frac{( TIC - {T}_{prep} )}{{T}_{1}}} - ( 1 - {M}_{startC} ) {e}^{- \frac{TRC - {T}_{read} - {T}_{prep}}{{T}_{1}}} ] \qquad ( 20 )$$

$$So \frac{{dM}_{endC}}{d {α}_{prep}} = [ {M}_{endC} + {e}^{- \frac{{T}_{pstartC}}{{T}_{1}}} - 1 ] \frac{1}{{α}_{prep}} \qquad ( 21 )$$

This can have the advantage that it is not directly sensitive to TI_{C} or TR_{C}.

Equating the two derivatives gives:

$$[ {M}_{end} + {e}^{- \frac{{T}_{pstartC}}{{T}_{1}}} - 1 ] \frac{1}{{α}_{prep}} = ( 1 - {M}_{startL} ) {e}^{- \frac{( TRL - {T}_{read} - {T}_{prep} )}{{T}_{1}}} - {e}^{- \frac{{T}_{pstartL}}{{T}_{1}}} \qquad ( 22 )$$

$${T}_{pstartC} = - {T}_{1} ln [ 1 - {M}_{end} + [ ( 1 - {M}_{startL} ) {e}^{- \frac{( TRL - {T}_{read} - {T}_{prep} )}{{T}_{1}}} - {e}^{- \frac{{T}_{pstartL}}{{T}_{1}}} ] {α}_{prep} ] \qquad ( 23 )$$

In some embodiments, given any T_{pstartL}, the T_{pstartC} can be calculated and the inversion times that will match the derivative of signal with respect to αₚᵣₑₚ for the two acquisitions (500, 600) can be determined.

In some embodiments, it may be also be desirable to match the two acquisitions (500, 600) for sensitivity to T₁. For example, to explore the sensitivity of the end magnetization to T₁ changes, it may be convenient to evaluate the equation for the derivative with respect to 1/T₁ numerically. Generally, it may be found that the T₁ dependence is small but almost perfectly matched for Tₚₛₜₐᵣₜₛ very close to the Tis. When the Tₚₛₜₐᵣₜₛ are moved away from the Tis, the sensitivity to T₁ may be found to be higher for the label image than the control image.

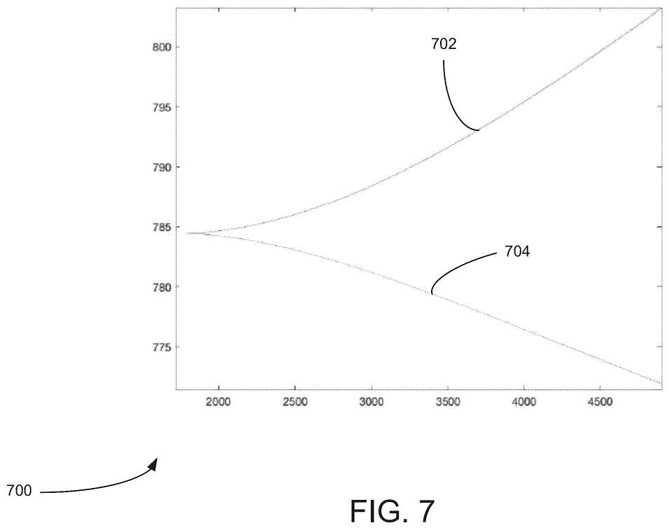

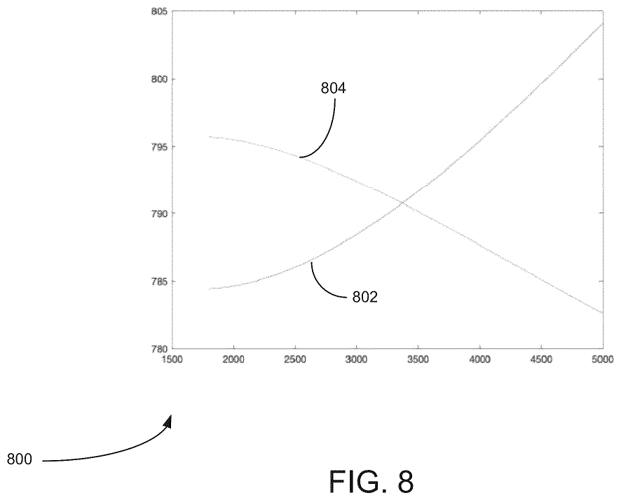

FIG. 7 shows an example graph illustrating a derivative with respect to 1/T₁ versus a time of the end of T₂-preparation in accordance with an embodiment. The graph 700 shows the derivative with respect to 1/T₁ for an example set of experimental parameters vs. T_{pstartL} (in ms). The top curve 702 shows the label image acquisition and the bottom curve 704 shows the control image acquisition. In some embodiments, optimal water exchange sensitivity comes with longer T_{pstartL}'s where the derivatives are not matched. The derivatives remain small, but to match the T₁ dependence at a particular T_{pstartL}, in some embodiments a different TR (e.g., a slightly longer TR) can be used for the control image acquisition 700. As mentioned above, using a different TR for the control image can reduce potential errors from differences in fluid T₁. FIG. 8 shows an example graph illustrating a derivative with respect to 1/T₁ versus a time of the end of T₂-preparation in accordance with an embodiment. The graph 800 is a similar plot to graph 700 in FIG. 7, but uses a different TR, in this example, a 50 ms longer TR, for the control image acquisition. Curve 802 shows the label image acquisition and the curve 804 shows the control image acquisition. In this example with a slightly longer TR, the T₁ dependence can be matched at a longer T_{pstartL}. In some embodiments, this may not be necessary for practical exchange imaging, given the relatively weak dependence on T₁. In some embodiments, the labeling efficiency between the two acquisitions can be evaluated to try to optimize the RI derivatives or signal.

The concepts discussed may be useful when the label and control image acquisition are actually sensitive to water exchange. It may be reasonable that the signal at the end of the acquisitions may differ if some of the magnetization in, for example, the CSF at the end originated from tissue where T₁ and T₂ are shorter but then exchanged into tissue.

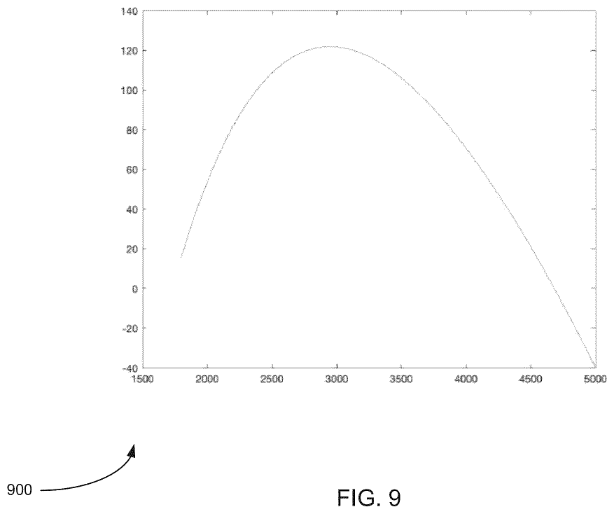

The simplest route for water exchange is that there are two large compartments, for example, CSF (fluid) and tissue (or choroid plexus). If these compartments are large and well mixed, then the effect of water exchange may be small and the exchange effect may be estimated to the first order assuming that the magnetization is in the tissue. The magnetization may first be determined as a function of time for tissue and CSF and then the small effect of exchange can be calculated according to the following differential equation:

$$\frac{{dM}_{1}^{csf}}{dt} = \frac{( 1 - {M}_{0}^{csf} ( t ) )}{{T}_{1}} - χ ( {M}_{0}^{csf} ( t ) - {M}_{o}^{tiss} ( t ) ) \qquad ( 24 )$$

Where χ is an exchange rate parameter. In some embodiments, this can be solved piecewise between RF pulses for different experiments. FIG. 9 shows an example graph illustrating sensitivity to water exchange as a function of a time of the end of T₂-preparation in accordance with an embodiment. Graph 900 shows the sensitivity to water exchange as a function of T_{pstartL}. Graph 900 illustrates that the sensitivity to exchange may be highest when there is considerable time between the prep and the inversion pulse. In the example shown in FIG. 9, the sensitivity peaks for a T_{pstartL} near 3000 ms.

Returning to FIG. 4, at block 404 the at least two images, e.g., a first image and a second image, may be generated (or reconstructed) based on the corresponding MR data for each image using known reconstruction methods. In some embodiments, the first image and the second image may be reconstructed using, for example, a data processing server 114 of an MRI system 100. In some embodiments, the first image and second image can be reconstructed using a computer system (e.g. computer system 1400 shown in FIG. 14) configured to access or receive the MR data acquired by the MR system. The generated first image and second image may be stored in, for example, data storage of an MR system (e.g., MRI system 100 shown in FIG. 1) or data storage of other computer systems.

At block 406, an image with signal representing water exchange may be generated by comparing the at least two images acquired at block 402 and generated at block 404, for example, comparing a first (or label) image to a second (or control) image. In some embodiments, the comparison may be implemented by subtracting the second image from the first image. In some embodiments, the comparison may be implemented by fitting the first and second images to a model or by using other comparison algorithms. The image with signal representing water exchange generated at block 406 may be stored in, for example, data storage of an MR system (e.g., MRI system 100 shown in FIG. 1) or data storage of other computer systems. At block 408, the image with signal representing water exchange may be used to determine an exchange signal fraction. An example of determining an exchange signal fraction for an example image with signal representing water exchange between CP and CSF of the brain is discussed further below. At block 410, water exchange may be quantified. For example, in some embodiments, the water exchange may be quantified using the generated image with signal representing water exchange, a reference image (e.g., acquired without T₂ preparation or inversion recovery), and a physical model. In some embodiments, quantification may be used to relate the exchange signal fraction to an exchange rate. In some embodiments, the quantified water exchange information may also be stored in, for example, data storage of the MRI system 100 shown in FIG. 1 or data storage of other computer systems.

At block 412, the generated image with signal representing water exchange and/or the quantified water exchange information may be displayed on a display, for example, a display of an MRI system (e.g., displays 104, 136 and/or 144 of MRI system 100 shown in FIG. 1) or a display of other computer systems.

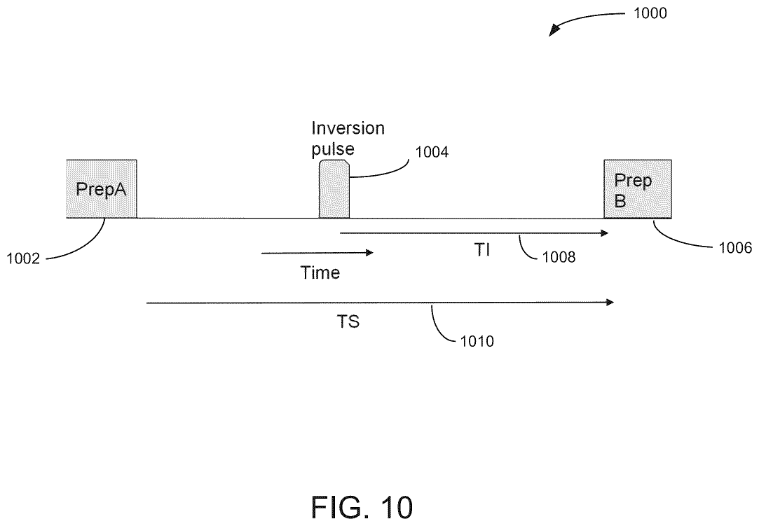

As discussed above with respect to FIGS. 2-9, the acquisition of the first and second images may include an inversion recovery (IR) pulse. In some embodiments, the pulse sequence may advantageously include a plurality of IR pulses (i.e., a plurality of inversions). While the following discussion of FIGS. 10-12 will refer to the acquisition of two images (e.g., a label image and a control image), it should be understood that in some embodiments, more than two images may be acquired and then compared to generate an image with signal representing water exchange.

FIG. 10 illustrates an example magnetization preparation sequence in accordance with an embodiment. In some embodiments, the magnetization preparation sequence illustrated in FIG. 10 generalizes the inversion recovery sequence described above. In FIG. 10, increasing time moves from left to right. Preparation A (PrepA) 1002 and preparation B (PrepB) 1006 may be, for example, T₂ prep sequences such as a BIR-8, MLEV, or other sequence for adding T₂ sensitivity. In some embodiments, PrepA 1002 and PrepB 1006 may have different T₂ sensitivity and one may even impart zero sensitivity by applying no RF pulses and having zero duration.

Now consider two different images acquired with different preparations. In some embodiments, in the first image (e.g., a label image), strong T₂ encoding may be applied for PrepA 1002 and short (or zero) T₂ encoding may be applied for PrepB 1006. In this case, the magnetization after the sequence may be given by:

$${M}_{afterA} = - α β {e}^{- \frac{TS}{{T}_{1}}} {M}_{start} + β ( 1 - 2 {e}^{- \frac{TI}{{T}_{1}}} + {e}^{- \frac{TS}{{T}_{1}}} ) \qquad ( 25 )$$

where Mₛₜₐᵣₜ is the tissue magnetization at the start of the sub sequence and T₁ is the tissue relaxation time, (1-alpha) is the (T₂ dependent) attenuation of magnetization due to PrepA 1002 and (1-beta) is the attenuation of PrepB 1006. For a second image (e.g., a control image), the preparations may be reversed with PrepB 1006 applied before TS (exchange time 1010) and PrepA 1002 applied after. The corresponding tissue magnetization may be given by:

$${M}_{afterB} = - α β {e}^{- \frac{TS}{{T}_{1}}} {M}_{start} + α ( 1 - 2 {e}^{- \frac{TI}{{T}_{1}}} + {e}^{- \frac{TS}{{T}_{1}}} ) \qquad ( 26 )$$

The difference between these two images (neglecting exchange effects) may be given by:

$${M}_{afterA} - {M}_{afterB} = ( β - α ) ( 1 - 2 {e}^{- \frac{TI}{{T}_{1}}} + {e}^{- \frac{TS}{{T}_{1}}} ) \qquad ( 27 )$$

For any given TS (exchange time) and T₁, an inversion time (TI 1008) may be chosen that will make the term in parentheses on the right of equation 27 zero. Thus, the difference of the tissue magnetization due to the inefficiencies contained in alpha and beta are eliminated. The difference will not be zero if magnetization exchanges between two compartments with different T₂/T₁. T₁ is not equal in all tissue, so this solution isn't perfect. For reasonably short TS (1010), the term on the right is still close to zero for a range of T₁'s. However, in some embodiments, the performance may be improved by adding more inversions. If we replace the single inversion (inversion pulse) 1004 with n inversions (n inversion pulses) between the two prep blocks 1002, 1006, the difference between the two images becomes:

$${M}_{afterA} - {M}_{afterB} = ( β - α ) ( 1 + {\sum }_{1}^{n} {( - 1 )}^{n} 2 {e}^{- \frac{{TI}_{n}}{{T}_{1}}} - {( - 1 )}^{n} {e}^{- \frac{TS}{{T}_{1}}} ) \qquad ( 28 )$$

In some embodiments, the choice of TI's can be selected (e.g. optimized) so the term on the right of equation 28 is less than 0.01 for a wide range of T₁'s. In practice, even two inversions (e.g., inversion (IR) pulses) can greatly reduce the T₁ sensitivity and can make the term less than 1% for most tissues. As a result, the sensitivity to the error term (β-α) can be reduced by a factor of more than 100. This can make it possible to measure exchange at more modest echo times (TE's) since total elimination of shorter T₂ tissue signal is not required.

FIG. 11 illustrates an example magnetization prepared inversion recovery pulse sequence configured to include a plurality of inversion recovery (IR) pulses in accordance with an embodiment. In FIG. 11, increasing time moves from left to right. In some embodiments, the example sequence 1100 of FIG. 11 may be used to acquire a first or label image. The pulse sequence 1100 shown in FIG. 11 includes a preparation module (PrepA) 1102 (for example, a T₂ preparation module), inversion 1104 which may include one or more inversion (IR) pulses (e.g., “n” IR pulse(s)), and a readout 1106. As discussed above, the magnetization preparation module 1102 may be applied before the inversion pulse(s) 1104. FIG. 12 illustrates an example magnetization prepared inversion recovery pulse sequence configured to include a plurality of inversion recovery (IR) pulses in accordance with an embodiment. In FIG. 12, increasing time moves from left to right. In some embodiments, the example sequence 1200 of FIG. 12 may be used to acquire a second or control image. The pulse sequence 1200 shown in FIG. 12 includes inversion 1202 which may include one or more inversion (IR) pulses (e.g., “n” IR pulse(s)), a magnetization preparation module (PrepA) 1204 (for example, a T₂ preparation module), and a readout 1206. As discussed above, the magnetization preparation module 1204 may be applied after the inversion pulse(s) 1202.

In FIGS. 11 and 12, the exchange time TS 1110, 1210 (corresponding to TS 1010 from FIG. 10) can be within the larger inversion recovery sequence. In the examples shown in FIGS. 11 and 12, PrepB is assumed to be a zero TE, no RF prep, so the conceptual PrepB is of zero duration and is invisible in the sequences. In this notation, the times (Tpre (1108, 1208) and Tpost (1112, 1212)) before and after the preparations and TS can be used to optimize contrast at the imaging time (for example, nulling CSF). Additional inversion pulses can be added to the exchange time TS (1110, 1210) for improved T₁ robustness as discussed above.

In the embodiments discussed above with respect to FIGS. 10-12, multiple inversion pulses can be applied to, for example, reduce errors from different tissue T₁'s. In some embodiments, the timing of the inversion pulse(s) can be selected (e.g., optimized) to reduce systematic errors over a range of T₁'s and T₂'s. Because the disclosed control strategy can work for a wide range of T₁'s, it is not necessary to strongly attenuate shorter T₂ species and in some embodiments more moderate and potentially even short TE's may be used. In some embodiments, better control of errors across T₂ and T₁'s of a tissue can make possible broader use of other types of contrasts such as, for example, velocity selective ASL, with the disclosed techniques for magnetization prepared MRI, as discussed further below.

Referring to FIGS. 10-12, in some embodiments, any preparation that attenuates longitudinal magnetization without allowing for recovery, i.e., whose effect can be approximated as reducing the longitudinal magnetization by a scale factor, can be used for the disclosed magnetization prepared MRI technique. For example, using BIR-8 preparations of fixed TE for both PrepA 1002, 1102, 1204 and PrepB 1006 but adding motion encoding gradients in PrepA 1002, 1102, 1204 to attenuate flowing spins could be used to selectively attenuate vascular spins. Exchange from the vasculature to the tissue over the exchange time TS (1010, 1110, 1210) would then be related to perfusion and this would be a new form of velocity selective ASL. A particular advantage of this control strategy can be that small eddy current or pulse imperfection errors would be compensated, unlike in other strategies, and systematic errors from any subject motion effects on the prep efficiency would be removed (though they still would be a potential source of noise). In some embodiments, virtually any MRI contrast could be imparted in these preparations (e.g., PrepA 1002, 1102, 1204) to reveal exchange or transport between tissues. For example, as mentioned, a velocity selective preparation can be used as the magnetization preparation module PrepA 1002, 1102, 1204 for performing velocity selective ASL imaging of perfusion.

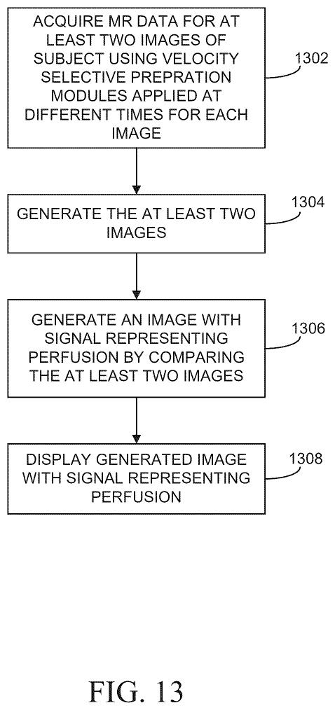

FIG. 13 illustrates a method for velocity selective arterial spin labeling magnetic resonance imaging of tissue perfusion in a region of interest in a subject in accordance with an embodiment. Although the blocks of the process in FIG. 4 are illustrated in a particular order, in some embodiments, one or more blocks may be executed in a different order than illustrated in FIG. 4 or may be bypassed.

At block 1302, MR data for at least two images may be acquired from a region of interest of a subject using, for example, an MRI system (e.g., MRI system 100 shown in FIG. 1). The MR data for each image may be acquired using a pulse sequence performed on the MRI system and the MR data for each image may be acquired with a different timing for the application of a velocity selective preparation. While the following discussion of FIG. 13 will refer to the acquisition and reconstruction of two images (e.g., a label image and a control image), it should be understood that in some embodiments, more than two images may be acquired, reconstructed, and then compared to generate an image with signal representing perfusion. In some embodiments, for example, MR data for a first (or label) image may be acquired from a subject using a pulse sequence that includes a velocity selective preparation module applied at a first predetermined time before a readout. In some embodiments, the first pulse sequence can include at least one inversion recovery pulse and the velocity selective preparation can be applied at a first time before the IR pulse(s). In some embodiments, the velocity selective preparation module may be implemented using known pulses and techniques for velocity selective preparation. In some embodiments, the preparation module can be any pulse that imparts a multiplicative change on longitudinal magnetization. The readout may be, for example, a three-dimensional fast spin echo readout.

At block 1302, MR data for a second image (or control image) may be acquired from a region of interest of a subject using a pulse sequence that includes a velocity selective preparation module applied at a second predetermined time before a readout where the second time is different than the first time. In some embodiments, the second pulse sequence can include at least one inversion recovery pulse and the velocity selective preparation can be applied at a second time after the IR pulse(s) and before the readout. In some embodiments, the velocity selective preparation module may be implemented using known pulses and techniques for velocity selective preparation. In some embodiments, the preparation module can be any pulse that imparts a multiplicative change on longitudinal magnetization. The readout may be, for example, a three-dimensional fast spin echo readout.

At block 1304 the at least two images, e.g., a first image and a second image, may be generated (or reconstructed) based on the corresponding MR data for each image using known reconstruction methods. In some embodiments, the first image and the second image may be reconstructed using, for example, a data processing server 114 of an MRI system 100. In some embodiments, the first image and second image can be reconstructed using a computer system (e.g. computer system 1400 shown in FIG. 14) configured to access or receive the MR data acquired by the MR system. The generated first image and second image may be stored in, for example, data storage of an MR system (e.g., MRI system 100 shown in FIG. 1) or data storage of other computer systems.

At block 1306, an image with signal representing perfusion may be generated by comparing the at least two images acquired at block 1302 and generated at block 1304, for example, comparing a first (or label) image to a second (or control) image. In some embodiments, the comparison may be implemented by subtracting the second image from the first image. In some embodiments, the comparison may be implemented by fitting the first and second images to a model or by using other comparison algorithms. The image with signal representing perfusion generated at block 1306 may be stored in, for example, data storage of an MR system (e.g., MRI system 100 shown in FIG. 1) or data storage of other computer systems. At block 1308, the generated image with signal representing perfusion may be displayed on a display, for example, a display of an MRI system (e.g., displays 104, 136 and/or 144 of MRI system 100 shown in FIG. 1) or a display of other computer systems.

The following examples set forth, in detail, ways in which the present disclosure was evaluated and ways in which the present disclosure may be used or implemented, and will enable one of ordinary skill in the art to more readily understand the principles thereof. The following examples are presented by way of illustration and are not meant to be limiting in any way.

### Example 1

As mentioned above, in some embodiments, the method for magnetic resonance imaging of tissue to fluid water exchange in a region of interest in a subject illustrated in FIG. 2 may be implemented to image a water exchange between CP and CSF of the brain. In this example study, water exchange between CP and CSF can be imaged and evaluated using a T₂-prepared, long TE Fluid Attenuated Inversion Recovery (FLAIR) sequence. Advantageously, in this example the disclosed method for imaging water exchange resulted in high SNR, high-resolution images of the choroid plexus and a signal difference determined between a T2-prepared image (i.e., the first, label image) and a control image (e.g., an image acquired without T2 preparation) was suggestive of CP-CSF water exchange.

In the absence of exchange, the CSF signal may be nulled by inversion timing and the CP signal may be effectively nulled by T₂ decay over a very long TE. However, magnetization that begins in the CP and exchanges to the CSF during the preparation time will not be fully nulled. If the water exchange is approximated as an immediate exchange of spins between the CSF and CP without any return, then the water exchange signal can be the integral over time before imaging of the difference between CP and CSF magnetization times the T₁ decay factor of CSF before imaging. An IR pulse can effectively invert the contribution before the IR pulse. When a T₂-preparation pulse is applied before the inversion pulse, shorter T₂ tissue including the CP can be essentially nulled. FIG. 14A shows an example graph 1402 illustrating magnetization as a function of time before readout for acquisitions with and without T₂ preparation in accordance with an embodiment and FIG. 14B shows an example graph 1404 illustrating magnetization difference between a tissue and a fluid with and without T₂ preparation in accordance with an embodiment. In FIG. 14A, graph 1402 illustrates the longitudinal magnetization (M_{z}) time evolution for CSF (curve 1406) and CP with (curve 1410) and without (curve 1408) T₂ preparation. For this example study, the graph 1404 in FIG. 14B illustrates the difference between the CP and CSF magnetization times the T₁ decay factor (with the difference reversed prior to the inversion pulse) with (curve 1414) and without (curve 1412) T₂ preparation. From graphs 1402 and 1404, it is apparent that without T₂-preparation, the exchange contribution after the inversion pulse approximately cancels the contribution from before. However, when T₂-preparation is applied, the positive contributions after the inversion pulse greatly increases resulting in a large net positive exchange signal.

In this example study, scans can be performed at 3T using, for example, a 48-ch head coil. In this example, 3D-FSE T₂-FLAIR data (TI/TR/TE=1785/6000/107 ms, ETL=220, linear view-ordering) was acquired, as well as a pair of long TE FLAIR acquisitions with and without T₂-preparation using reversed-centric view-ordering, with 25 discarded echoes to avoid propagation of high-frequency features during early echoes, ETL=245, TR/TE-6/1 s, first refocusing flip angle of 120 degrees followed by a gradual ramp down to 75 degrees. TI may be, for example, automatically adjusted on a case-by-case basis depending on echo-train duration with a null target of T1=4.27 s taking into account the T₂-preparation (TI≈1737-1770 ms). An additional reference volume without IR or T₂ preparation can be acquired to serve as a reference image.

In this example study, the T₂-preparation can consist of a 90 degrees hard pulse, followed by 4 adiabatic hyperbolic-secant refocusing pulses and one final-90 degrees hard pulse. As mentioned, a second, separate FLAIR volume without T₂-preparation can also be acquired and adjusted TI for CSF nulling without T₂-preparation (TI≈1850-1890 ms). In this example study, common parameters were: 136 sagittal slices, matrix=192×192 leading to (1.3 mm) 3 resolution, parallel imaging with 2×2 acceleration in both phase-encoded directions for an acquisition time of 3 min 21 s per volume (total scan time 16.5 min).

The first (label) image with T₂ preparation and the second (control) image without T₂ preparation can be reconstructed using known methods, which can be followed by subtraction between the T2-prepared (label) and the control volumes (or images). An exchange signal fraction (ESF) can be calculated as:

$$ESF = \frac{{T}_{2 {P}_{on}} ( CP ) - {T}_{2 {P}_{off}} ( CP )}{Ref ( CSF )} \qquad ( 29 )$$

with CP and CSF corresponding to the mean value in a ROI (region of interest) positioned in the choroid plexus and neighboring CSF respectively.

In this example study it was shown that the signal in the CP is substantially higher in the T₂-prepared volume (or image) compared to the control volume (or image). In this example study, a subtraction between the T₂-prepared and control volumes showed higher signal in the CP compared to surrounding CSF. The subtraction experiment can allow for controlling for potential remaining T₂ signal in the CP, but the imperfect CSF nulling in the control volume leads to substantial CSF contamination in the subtraction image. The average ESF was found equal to 2.2±0.4%.

In some embodiments, T₂-prepared data can be acquired with significantly longer TE of 1.8 s to eliminate any possibility of residual CP signal contamination. The presence of a strong remaining signal in the CP at such TE confirms water exchange as the source of the strong signal in the CP.

This example study illustrated evidence of water exchange in the choroid plexus using T₂-prepared, long TE FLAIR imaging. This study produced high-resolution images of the CP and could allow, for example, for studying CP function, complementary to other methods such as ASL that has recently gained traction for the study of CP function. In some embodiments, a control may be provided that adequately matches CSF signal levels to the T₂-prepared acquisition in order to support quantification. The quantification could be used for various applications such as, for example, in pathology such as Alzheimer's disease.

### Example 2

Alterations of renal water transport and exchange may reflect kidney function and disease. In some embodiments, the method for magnetic resonance imaging of tissue to fluid water exchange in a region of interest in a subject illustrated in FIG. 4 may be implemented for quantitative assessment of intrarenal fluid exchange. In this example study, images showed a characteristic spatial distribution of signal with increased signal in the renal medulla, in support of its detection and assessment of fluid exchange. Accordingly, the disclosed technique for MR imaging may enable studies of intrarenal fluid exchange and potentially serve as a biomarker for diagnosis and prognosis of renal diseases.

As discussed above with respect to FIGS. 4-6 and 11-12, the disclosed technique can be used for T₂ selective labeling that employs, in some embodiments, a second image with T₂ saturation (i.e., a T₂ preparation) applied at a different time (e.g., later) as a control. In this example study, a first (or label) image was acquired from a subject using a pulse sequence that included a T₂ preparation module applied at a first predetermined time before a readout and a second (or control) image was acquired from the subject using a pulse sequence that included a T₂ preparation module applied at a second predetermined time before a readout where the second time is different than the first time, for example, the T₂ preparation for the second image can be applied at a later time. For example, the pulse sequence of the first or label image, a T₂ selective saturation (i.e., a T₂ preparation) can be applied before a mixing time, Tₘᵢₓ, (i.e., an exchange time such as TS referred to above) and the pulse sequence of the control image, the T₂ selective saturation can be applied at a different time, for example, after the mixing time. In the absence of exchange during the mixing time, the magnetization after the label and control sequences may be given by M_{2lbl} and M_{2ctl}, respectively:

$${M}_{2 lbl} = α {M}_{1} ( {e}^{( - {T}_{mix} / {T}_{1} )} ) + R \qquad ( 30 )$$

$${M}_{2 ctl} = α {M}_{1} ( {e}^{( - {T}_{mix} / {T}_{1} )} ) + α R \qquad ( 31 )$$

where α is the saturation factor for the T₂ selective saturation and R is the recovered magnetization during the mixing period. The difference between the two ending magnetizations is nonzero only because of the recovery term R, as given by:

$${M}_{2 lbl} - {M}_{2 ctl} = ( 1 - α ) R = ( 1 - α ) ( 1 - {e}^{( - {T}_{mix} / {T}_{1} )} ) \qquad ( 32 )$$

If n inversion pulses (or inversions) are added during the mixing time (or exchange time), the M₁ terms in equations 30-32 above are simply multiplied by a power of the inversion efficiency factor. The timing of the inversion pulses can be selected (e.g., optimized) so that R is close to zero. For three or more inversion pulses (or inversions), R can be reduced to, for example, less than 1% for T1's from pure water to fat.

$${M}_{2 lbl} - {M}_{2 ctl} = {αβ}^{n} {M}_{1} {e}^{( - {T}_{mix} / {T}_{1} )} - {αβ}^{n} {M}_{1} {e}^{( - {T}_{mix} / {T}_{1} )} + ( 1 - α ) R \qquad ( 33 )$$

Accordingly, the direct effects of the label can be matched by the control. As discussed above, any differences between the label image and the control image can reflect exchange during the exchange or mixing time, for example, such that T₂ and/or T₁ are not the same for exchanging spins across the measured region.

In this example study, the label and control images can be acquired using a 2D SSFSE (RARE) sequence. T₂ preparation can be implemented with, for example, either 100 ms or 200 ms BIR8 adiabatic sequences, and 4 tanh adiabatic inversion pulses can be applied at optimized times to minimize recovered magnetization. In this example, the T₂ preparations were preceded by nonselective saturation 5 s before imaging and a T₂ inversion recovery optimized to nearly nullify M1 of renal fluid. Following the T₂ preparations, in this example 200 ms were allowed to allow some recovery of tissue magnetization and 3 fat saturation pulses were applied immediately before imaging. A TR of 10 s with interleaving of label and control acquisitions and variable TE's were used in this example. TE can be controlled by skipping a number of echoes prior to acquisition. In this example, eleven acquisitions of the label and control images and a reference image required a total of 4 minutes per sequence. In this example, the label images were averaged and the control images were averaged, and then the averaged control images were subtracted from the averaged label images to create an image with signal representing water exchange.

In this example, images showed predominantly increased signal within the renal medulla and distributed throughout the medullary collecting duct system especially at longer labeling times. Cortical signals were relatively more pronounced at shorter labeling times (exchange or mixing time of 1000 ms). This signal's spatial distribution could be explained by a mixture of water exchange in the proximal tubule and collecting duct and the bulk flow of filtrate. This example study illustrated that the disclosed method can provide an approach for sensitive measurement of exchange from short to long TE compartments that can be used to assess water exchange within the kidney. The disclosed technique can provide a non-invasive assessment of renal filtration and water exchange that may complement other measures of renal function.

### Example 3

As mentioned above, water exchange between tissue and CSF may contribute to CSF production and glymphatic clearance. The large difference in T₂ between tissue and fluid suggests T₂ magnetization transfer can be used to image this exchange. In some embodiments, the method for magnetic resonance imaging of tissue to fluid water exchange in a region of interest in a subject illustrated in FIG. 4 may be implemented for studies of CSF exchange that may reflect change in glymphatic clearance or CSF production with aging, Alzheimer's disease, intracranial hypertension and other disorders. As mentioned above, the disclosed technique can advantageously be used to control for systematic errors from direct effects of T₂ saturation on fluid. In this example study, three dimensional images at longer TE can show exchange signal surrounding the choroid plexus, and can also show more modest exchange near the cerebellar vermis and the cerebellar and cerebral cortices.

As discussed above with respect to FIGS. 4-6 and 11-12, the disclosed technique can be used for T₂ selective labeling that employs, in some embodiments, a second image with T₂ saturation (i.e., a T₂ preparation) applied at a different time (e.g., later) as a control. In this example study, a first (or label) image was acquired from a subject using a pulse sequence that included a T₂ preparation module applied at a first predetermined time before a readout and a second (or control) image was acquired from the subject using a pulse sequence that included a T₂ preparation module applied at a second predetermined time before a readout where the second time is different than the first time, for example, the T₂ preparation for the second image can be applied at a later time. For example, the pulse sequence of the first or label image, a T₂ selective saturation (i.e., a T₂ preparation) can be applied before an mixing time, Tₘᵢₓ, (i.e., an exchange time such as TS referred to above) and the pulse sequence of the control image, the T₂ selective saturation can be applied at a different time, for example, after the mixing time. In the absence of exchange during the mixing time, the magnetization after the label and control sequences may be given by M_{2lbl} and M_{2ctl}, respectively, as shown in Equations 30 and 31 above. The difference between the two ending magnetizations is nonzero only because of the recovery term R and can be given by Equation 32 above. If n inversion pulses (or inversions) are added during the mixing time (or exchange time), the M₁ terms in equations 30-32 above are simply multiplied by a power of the inversion efficiency factor. The timing of the inversion pulses can be selected (e.g., optimized) so that R is close to zero. For three or more inversion pulses (or inversions), R can be reduced to, for example, less than 1% for T₁'s from pure water to fat (see Equation 33 above). As discussed above, because the subtraction can remove any direct effects of labeling on exchanging spins, any differences between the label image and the control image can reflect exchange during the exchange or mixing time, for example, such that T₂ and/or T₁ are not the same.

In this example study, the label and control images can be acquired using a 3DFSE (RARE) sequence. In this example, T₂ preparation can be implemented with a 200 ms TE BIR8 adiabatic sequence, and 4 tanh adiabatic inversion pulses can be applied at optimized times to minimize recovered magnetization. The T₂ preparations can be preceded by nonselective saturation 5 s before imaging and a T₂ selective inversion recovery can be optimized to nearly null CSF M₁. Following the T₂ preparations, 200 ms were allowed to allow some recovery of tissue magnetization and 3 fat saturation pulses were applied immediately before imaging. In this example, a TR of 10 s, 2×2 parallel imaging acceleration, an asymptotic 70° flip angle train with echo spacing of 3.3 ms, and centric phase ordering with TE controlled by skipping echoes prior to acquisition were elected. In this example, acquisition of the label and control images and an unprepared reference image required 5 min 20 s. In this example, images were acquired in 3 healthy volunteers and for TE's of 106.5, 213.0 and 319.5 ms for Tₘᵢₓ of 2 s and 1.5 s and for the 2 longer TE's at Tₘᵢₓ of 1 s. Following gaussian smoothing to 3×3×3 mm resolution, in this example study label and control images were subtracted and divided by the signal in the center of the ventricles on the reference image.

In this example study, all images showed elevated signal surrounding the choroid plexus and distributed throughout cortical and brain stem regions. Negative white matter signal was noticeably present on the TE 106.5 ms images but the effect faded by the 213 ms images and was negligible in the 319.5 ms images. This effect likely reflects incomplete suppression of recovered magnetization due to a very short T₁ component in white matter. Exchange signal in choroid plexus and near cortex appeared to increase slightly with TE, consistent with reduced partial volume of blurred negative white matter signal. Though the exchange signal was present only in regions known to contain CSF, this signal could not simply be a systematic error in CSF, since the spatial variation of intensity was very different from the unsubtracted label or control images and the reference images. 3D images averaged across subjects show the whole brain distribution of the exchange signal. In this example study, the spatial distribution of exchange signal was consistent across subjects with the highest signal around the choroid plexus of the lateral ventricles. Signal was also prominent in the fourth ventricle and around the cerebellar vermis. In this example, noticeable exchange may be seen surrounding the cerebellar and cerebral cortices.

This example study illustrated that the disclosed method can provide an approach for sensitive measurement of exchange from short to long T2 compartments that can be used to assess water exchange from tissue and blood to CSF. The disclosed may be used to help understand and diagnose disorders of CSF production and the glymphatic clearance system.

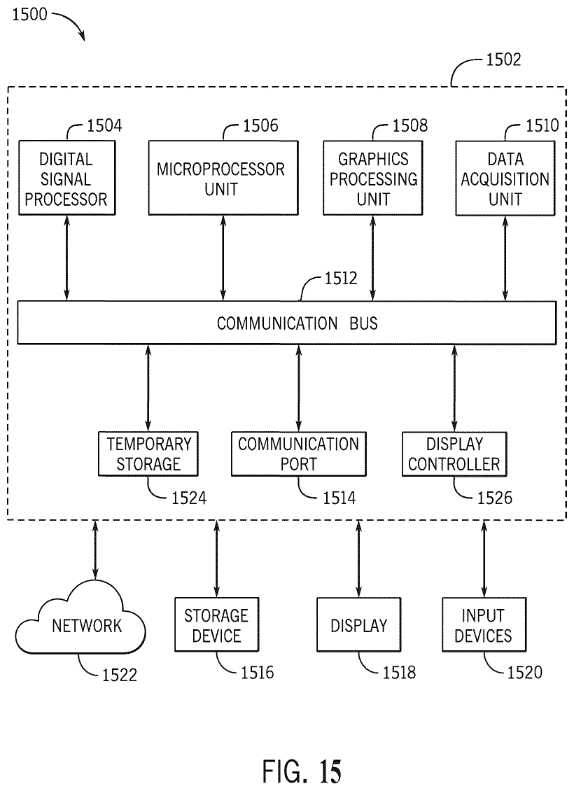

FIG. 15 is a block diagram of an example computer system in accordance with an embodiment. Computer system 1500 may be used to implement aspects of the systems and methods described herein. In some embodiments, the computer system 1500 may be a workstation, a notebook computer, a tablet device, a mobile device, a multimedia device, a network server, a mainframe, one or more controllers, one or more microcontrollers, or any other general-purpose or application-specific computing device. The computer system 1500 may operate autonomously or semi-autonomously, or may read executable software instructions from the memory or storage device 1516 or a computer-readable medium (e.g., a hard drive, a CD-ROM, flash memory), or may receive instructions via the input device 1520 from a user, or any other source logically connected to a computer or device, such as another networked computer or server. Thus, in some embodiments, the computer system 1500 can also include any suitable device for reading computer-readable storage media.

Data, such as data acquired with, for example, an imaging system (e.g., a magnetic resonance imaging (MRI) system, etc.), may be provided to the computer system 1500 from a data storage device 1516, and these data are received in a processing unit 1502. In some embodiments, the processing unit 1502 included one or more processors. For example, the processing unit 1502 may include one or more of a digital signal processor (DSP) 1504, a microprocessor unit (MPU) 1506, and a graphic processing unit (GPU) 1508. The processing unit 1502 also includes a data acquisition unit 1510 that is configured to electronically receive data to be processed. The DSP 1503, MPU 1506, GPU 1508, and data acquisition unit 1510 are all coupled to a communication bus 1512. The communication bus 1512 may be, for example, a group of wires, or a hardware used for switching data between the peripherals or between any component in the processing unit 1502.

The processing unit 1502 may also include a communication port 1514 in electronic communication with other devices, which may include a storage device 1516, a display 1518, and one or more input devices 1520. Examples of an input device 1520 include, but are not limited to, a keyboard, a mouse, and a touch screen through which a user can provide an input. The storage device 1516 may be configured to store data, which may include data such as, for example, MR data, MR images (e.g. label image, control images, images with signal representing water exchange), etc., whether these data are provided to, or processed by, the processing unit 1502. The display 1518 may be used to display images and other information, such as patient health data, and so on.

The processing unit 1502 can also be in electronic communication with a network 1522 to transmit and receive data and other information. The communication port 1514 can also be coupled to the processing unit 1502 through a switched central resource, for example the communication bus 1512. The processing unit 1502 can also include temporary storage 1524 and a display controller 1526. The temporary storage 1524 is configured to store temporary information. For example, the temporary storage can be a random access memory.

Computer-executable instructions for magnetization prepared magnetic resonance imaging according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access

The present invention has been described in terms of one or more preferred embodiments, and it should be appreciated that many equivalents, alternatives, variations, and modifications, aside from those expressly stated, are possible and within the scope of the invention.

1. A method for magnetic resonance imaging of tissue to fluid water exchange in a region of interest in a subject, the method comprising:
acquiring, using a magnetic resonance imaging (MRI) system, magnetic resonance (MR) data for at least two images of the region of interest of the subject using a different T₂ preparation for each image;
generating the at least two images using the corresponding MR data; and
generating an image with signal representing water exchange by comparing the at least two images.

2. The method according to claim 1, wherein acquiring MR data for the at least two images of the region of interest of the subject comprises:
acquiring MR data for a first image of the region of interest of the subject using a first pulse sequence comprising a T₂ preparation; and
acquiring MR data for a second image of the region of interest of the subject using a second pulse sequence without a T₂ preparation.

3. The method according to claim 1, wherein acquiring MR data for the at least two images of the region of interest of the subject comprises:
acquiring MR data for a first image of the region of interest of the subject using a first pulse sequence comprising a T₂ preparation with a first duration; and
acquiring MR data for a second image of the region of interest of the subject using a second pulse sequence comprising a T₂ preparation with a second duration.

4. The method according to claim 2, wherein the first pulse sequence and the second pulse sequence further comprise at least one inversion recovery (IR) pulse.

5. The method according to claim 3, wherein the first pulse sequence and the second pulse sequence further comprise at least one inversion recovery (IR) pulse.

6. The method according to claim 1, wherein comparing the at least two images comprises subtracting at least one of the at least two images from at least one of the other images.

7. The method according to claim 6, further comprising:
retrieving a reference image acquired without T₂ preparation and inversion recovery;
calculating an exchange signal fraction based on the reference image and the subtraction of the at least two images.

8. The method according to claim 1, further comprising displaying the image with signal representing water exchange.

9. The method according to claim 2, wherein the first pulse sequence and the second pulse sequence further comprise a readout with a long echo time.

10. The method according to claim 3, wherein the first pulse sequence and the second pulse sequence further comprise a readout with a long echo time.

11. The method according to claim 1, wherein the region of interest is a brain of the subject.

12. The method according to claim 1, wherein the region of interest is a kidney of the subject.

13. A method for magnetic resonance imaging of tissue to fluid water exchange in a region of interest in a subject, the method comprising:
acquiring, using a magnetic resonance imaging (MRI) system, magnetic resonance (MR) data for at least two images of the region of interest of the subject using a different timing for application of a T₂ preparation for each image;
generating the at least two images using the corresponding MR data; and
generating an image with signal representing water exchange by comparing the at least two images.

14. The method according to claim 13, wherein acquiring MR data for the at least two images of the region of interest of the subject comprises:
acquiring MR data for a first image of the region of interest of the subject using a first pulse sequence comprising a T₂ preparation applied at a first time; and
acquiring MR data for a second image of the region of interest of the subject using a second pulse sequence comprising a T₂ preparation applied at a second time, wherein the second time is different than the first time.

15. The method according to claim 14, wherein the first pulse sequence further comprises at least one inversion recovery (IR) pulse and the first time is before the at least one IR pulse.

16. The method according to claim 14, wherein the second pulse sequence further comprises at least one inversion recovery (IR) pulse and the second time is after the at least one IR pulse.

17. The method according to claim 13, wherein comparing the at least two images comprises subtracting at least one of the at least two images from at least one of the other images.

18. The method according to claim 13, wherein acquiring MR data for the at least two images of the region of interest of the subject comprises:
acquiring MR data for a first image using a first pulse sequence comprising a first repetition time (TR); and
acquiring MR data for a second image using a second pulse sequence comprising a second repetition time (TR), wherein the second TR is different than the first TR.

19. The method according to claim 13, further comprising quantifying water exchange based on the image with signal representing water exchange and a physical model.

20. The method according to claim 13, further comprising displaying the image with signal representing water exchange.

21. The method according to claim 13, wherein acquiring MR data for the at least two images of the region of interest of the subject comprises:
acquiring MR data for a first image using a first pulse sequence comprising a readout with a long echo time; and
acquiring MR data for a second image using a second pulse sequence comprising a readout with a long echo time.

22. The method according to claim 13, wherein the region of interest is a brain of the subject.

23. The method according to claim 13, wherein the region of interest is a kidney of the subject.

24. A magnetic resonance imaging (MRI) system comprising:
a magnet system configured to generate a polarizing magnetic field about at least a portion of a subject;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance signals from the subject using a coil array; and
a computer system programmed to:
acquire magnetic resonance (MR) data for at least two images of the region of interest of the subject using a different T₂ preparation for each image;
generate the at least two images using the corresponding MR data; and
generate an image with signal representing water exchange by comparing the at least two images.

25. A magnetic resonance imaging (MRI) system comprising:
a magnet system configured to generate a polarizing magnetic field about at least a portion of a subject;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance signals from the subject using a coil array; and
a computer system programmed to:
acquire magnetic resonance (MR) date for at least two images of the region of interest of the subject using a different timing for application of a T₂ preparation for each image;
generate the at least two images using the corresponding MR data; and
generate an image with signal representing water exchange by comparing the at least two images.

26. A method for velocity selective arterial spin labeling magnetic resonance imaging of tissue perfusion in a region of interest in a subject, the method comprising:
acquiring, using a magnetic resonance imaging (MRI) system, magnetic resonance (MR) data for at least two images of the region of interest of the subject using a different timing for application of a velocity selective preparation for each image;
generating the at least two images using the corresponding MR data; and
generating an image with signal representing perfusion by comparing the at least two images.

27. The method according to claim 26, wherein acquiring MR data for the at least two images of the region of interest of the subject comprises:
acquiring MR data for a first image of the region of interest of the subject using a first pulse sequence comprising a velocity selective preparation applied at a first time; and
acquiring MR data for a second image of the region of interest of the subject using a second pulse sequence comprising a velocity selective preparation applied at a second time, wherein the second time is different than the first time.

28. The method according to claim 27, wherein the first pulse sequence further comprises at least one inversion recovery (IR) pulse and the first time is before the at least one IR pulse.

29. The method according to claim 27, wherein the second pulse sequence further comprises at least one inversion recovery (IR) pulse and the second time is after the at least one IR pulse.

30. The method according to claim 26, wherein comparing the at least two images comprises subtracting at least one of the at least two images from at least one of the other images.
